(19) United States
(12) Patent Application Publication
(10) Pub. No.: US 2021/0332247 A1
(43) Pub. Date: Oct. 28, 2021

(54) CHAMPAGNE-TONE METAL EFFECT PIGMENTS

(71) Applicant: SCHLENK METALLIC PIGMENTS GMBH, Roth (DE)

(72) Inventors: Yvonne BINDER, Buchenbach (DE); Adalbert HUBER, Bencheim (DE); Daniel GRASER, Schwabach (DE)

(21) Appl. No.: 17/269,556

(22) PCT Filed: Jul. 25, 2019
(86) PCT No.: PCT/EP2019/070095

§ 371 Date: Feb. 19, 2021

(30) Foreign Application Priority Data
Aug. 22, 2018 (EP) 18190262.8

(51) Int. Cl. C09C 1/00; C09D 5/36; C09D 7/40; C09D 7/62; C09D 11/50; C09D 11/037; C03C 1/04; A61K 8/26; A61K 8/02; A61K 8/25; A61K 8/19; A61Q 1/02

(52) U.S. Cl. CPC C09C 1/0066; A61K 2800/63; C09D 5/36; C09D 7/70; C09D 7/62; C09D 11/50; C09D 11/037; C03C 1/04; C09C 1/0009; A61K 8/26; A61K 8/0266; A61K 8/25; A61K 8/19; A61Q 1/02; C09C 2200/1058; C09C 2200/301; C09C 2200/303; C09C 2200/302; C09C 2220/106; A61K 2800/413; A61K 2800/43; A61K 2800/651; C09C 1/0075

(57) ABSTRACT

The present invention relates to metal effect pigments with a high covering power and a thin iron oxide coating, for creating a champagne tone.

The present invention relates to metallic effect pigments with high hiding power and a thin iron oxide layer for generating a champagne-color tone.

To date there are no known comparable metallic effect pigments or pearlescent pigments that have a high hiding power, a yellowish-beige tone with a hue angle (hᵤᵥ) in the range of 50-65 (champagne-tone), and a saturation (Sᵤᵥ) in the range of 0.2-0.3 (for a ΔE45° of ≤3). Pigments that are currently on the market and have similar tones, such as ALOXAL® from Eckart GmbH or Iriodin 9602 from Merck, for example, have a hiding power which is sometimes unsatisfactory and/or have significantly deviating color tones and color saturation values.

EP 2 999 752 B1 discloses metallic effect pigments based on thin Al substrate platelets from a VMP process, with high hiding power and with Fe₂O₃ as a coloring layer. WO 2013/175339 discloses aluminum-based metallic effect pigments having at least one metal oxide layer. EP 2 303 969 B1 discloses the production of mixtures of iron and aluminum effect pigments. WO 2015/040537 A1 discloses nonmagnetizable effect pigments consisting of an aluminum or aluminum alloy core with one or more passivation layers and an aluminum-doped iron oxide layer. WO 2007/093334 A1 discloses a method for producing color effect pigments and the use thereof in cosmetics. These colored effect pigments consist of an aluminum-containing core, surrounded by a coloring layer, which is generated by wet-chemical oxidation of the Al core, and/or an effect pigment with at least one metal oxide layer which comprises at least one color pigment. CA 2,496,126 discloses effect pigments based on metals (silver, gold, copper, aluminum, zinc, titanium, and alloys thereof) for use in cosmetics, which are coated with a further metal oxide layer in a wet-chemical method by way of a sol-gel process. U.S. Pat. No. 7,828,890 B2 discloses effect pigments having an aluminum or aluminum alloy core and, enveloping the aluminum or aluminum alloy core, an aluminum oxide-containing or aluminum oxide/hydroxide-containing layer, obtainable by wet-chemical oxidation of platelet-shaped aluminum or aluminum alloy pigments, where the amount of metallic aluminum in the aluminum or aluminum alloy core is not more than 90 wt %, based on the total weight of the pigment, where the oxidized aluminum or aluminum alloy pigments have at least one high-index metal chalcogenide layer having a refractive index of >1.95, and, formed between the high-index metal chalcogenide layer and the enveloping aluminum oxide-containing or aluminum oxide/hydroxide-containing layer, a mixed layer. EP 0 950 693 A1 discloses multilayer pearlescent pigments based on a nonmetallic substrate. WO 2011/085780 A1 discloses multilayer effect pigments based on nonmetallic substrates. WO 96/22336 A1 discloses colored pigments based on platelet-shaped aluminum substrates, coated with a metal oxide layer, having color pigments in the metal oxide layer. The specialist book “Metalleffekt-Pigmente” (2^{nd} revised edn., Peter Willing et al., Vincentz-Verlag) sets out, in Part III—Specialty Aluminum Pigments, in Chapter 2 Colored Aluminum Pigments (pp. 70-76), the production of an oxidized aluminum pigment with “champagne-colored” appearance (trade name ALOXAL®) and the use thereof in combination with colored pigments. Similarly to this, DE 195 20 312 B4 discloses the production of colored effect pigments by wet-chemical oxidation of aluminum platelets.

EP 3 345 974 A1 and EP 3 081 601 A1 do describe metallic effect pigments or pearlescent pigments, but not those having a yellowish-beige tone with a hue angle (hᵤᵥ) in the range of 50-65 (champagne tone) and also a saturation (Sᵤᵥ) in the range of 0.2-0.3 (for a ΔE45° of ≤3).

The object of the present invention is therefore to provide metallic effect pigments having a high hiding power and a yellowish-beige tone with a hue angle (hᵤᵥ) in the range of 50-65 (champagne tone) and also a saturation (Sᵤᵥ) in the range of 0.2-0.3 (for a ΔE45° of ≤3).

This object is achieved by the embodiments characterized in the claims.

Provided more particularly is a metallic effect pigment based on coated aluminum substrate platelets, where the aluminum substrate platelets have a thickness of 5 to 35 nm, preferably of 15 to 25 nm, are monolithic in construction, and are surrounded by a “native” passivation layer—that is, a passivation layer resulting from the production process but not applied in a separate process step—of Al₂O₃ of less than 10 nm, typically 2 to 5, generally 2 to 3 nm, where these aluminum substrate platelets are enveloped by at least one coating A of SiO₂ having a layer thickness of 10 to 100 nm, preferably 20 to 80 nm, bearing a further, applied coating B of Fe₂O₃ which envelops the substrate platelets with the SiO₂ coating A and which has a layer thickness of 5 to 45 nm, preferably 10 to 45 nm, more preferably 20 to 45 nm, more particularly 20 to 40 nm, and characterized, for a ΔE45 of ≤3 at not more than 7% pigmentation, more particularly ΔE45° of ≤1 at <7% pigmentation, by a saturation (Sᵤᵥ) in the range of 0.2-0.3, a value for the hue angle (hᵤᵥ) in the range of 50-65, a value for the flop/Alman Index above 26, and a value for the Gdiff (graininess) at not more than 6.

Provided optionally in addition may be a further, outer layer C, which in turn envelops the coating B, for example, an SiO₂ layer 1 to 30 nm thick, which optionally may also penetrate the lower Fe₂O₃. Moreover, an organic or organic-inorganic surface modification may be applied to the layer B or to such a layer C, if present.

The SiO₂ layer A, which may be regarded as the actual passivating layer, is applied wet-chemically via a sol-gel process. The layer B consists of Fe₂O₃ applied wet-chemically via a precipitation reaction, having a layer thickness of 5 to 45 nm, preferably 10 to 45 nm, more preferably 20 to 45 nm, more particularly 20 to 40 nm. The iron oxide layer may also be a mixed layer, which comprises one or both of the metal oxides, selected from Al₂O₃ or TiO₂, in a (total) fraction of more than 0 to 20 wt %, based on the layer B.

The aluminum substrate platelets are polycrystalline and have an average thickness of 5 to 35 nm, preferably of 15 to 25 nm. Preferably each substrate platelet has an extremely uniform thickness. As a result of the production process, however, there may be fluctuations in the thickness within a platelet. These fluctuations ought preferably not to be more than ±25%, based on the average thickness of the platelet in question, more preferably at most ±10%. The average thickness here is the numerical mean of maximum and minimum thickness. Determining the minimum and maximum layer thickness is done by measurements on the basis of transmission and scanning electron micrographs (TEM and SEM) of coated substrate platelets.

Aluminum platelets used are preferably those known as vacuum metallized pigments (VMPs). VMPs can be obtained by detaching aluminum from metallized films. They are notable for particularly low substrate platelet thickness and for a particularly smooth surface with increased reflectivity. Platelets or flakes in the context of the present invention are those having a thickness/length ratio of at least 1:100, preferably higher.

Any reference here to the “thickness” of a coating or of an aluminum substrate platelet shall be understood as referring to the average thickness as designated, unless something different is defined at the relevant point.

The aluminum substrate platelets are monolithic in construction. Monolithic in this context means that they consist of a single self-contained unit without fractures, layers or inclusions, although there may be changes in microstructure within the substrate platelets. The aluminum substrate platelets are preferably homogeneous in construction, meaning that there is no concentration gradient within the platelets. In particular, the aluminum substrate platelets are not layerlike in construction (the “native” Al₂O₃ passivation layer, resulting from the production process but not applied in a separate process step, is disregarded in this context) and they have no particles distributed in them. In particular they do not have core-shell construction, with the shell consisting, for example, of a material suitable for the substrate platelets, and the core consisting of a different material, such as a silicon oxide, for example. In contrast to this, a more complex, non-monolithic construction on the part of the substrate platelets implies a more difficult, time-consuming and costly production process.

The percentage figures in the subsequent sections should be read, unless otherwise noted, as wt %.

When knife-coated out (hidingly) with ΔE45°≤1, the metallic effect pigments of the invention exhibit values for the saturation (Sᵤᵥ) in the range of 0.2-0.3, values for the hue angle (hᵤᵥ) in the range of 50-65, values for the flop/Alman Index above 26, and values for the Gdiff (graininess) at not more than 6. The very good hiding power of the metallic effect pigments of the invention is evident for the fact that for knife-applied hiding coatings, defined here by a ΔE45°≤1, pigmentations of not more than 7% are required. In particular, the values for ΔE45° for 7% pigmentation are below 3.0. A color tone of this kind with corresponding hiding power and flop cannot be achieved through mixtures of metallic effect pigments with organic and/or inorganic pigments. Furthermore, pigment mixtures are less precise in terms of the reproduction of the color tone setting.

In contrast to pigment mixtures based on aluminum pigments, the metallic effect pigments of the invention are not combustible and therefore do not have to be classed as hazardous goods for the purpose of transportation, for example.

Conversely, for example, the noninventive example of the commercially available pigment Merck Iriodin 9602 fails to achieve the aforementioned values for the ΔE45° hiding power even at high levels of pigmentation. A hiding power with a value of ΔE45°<3 cannot be achieved even at very high pigmentations of 20% (ΔE45 here is 5.1). A knife-applied hiding coating with ΔE45°≤1 cannot be anticipated, arithmetically, until the pigmentation is >30%.

If the commercially available pigment Iriodin 9602 with a pigmentation of 20% is mixed with a yellow pigment Helio Beit UN210 (4.5%; initial masses of the yellow pigment, unless otherwise characterized, based on the dry mass of the pigment used), in an attempt to imitate a champagne tone, the value of the ΔE45° hiding power of this mixture is situated in the upper region of the range achieved by the metallic effect pigments of the invention, but here the significantly higher pigmentation must be borne in mind. The value for the saturation of the mixture is within the range of 0.2-0.3 achieved by the metallic effect pigments of the invention, although a composition of this kind is unable to attain the hue angle (hᵤᵥ) achieved by the metallic effect pigments of the invention or the defined flop value (here, the addition of the yellow paste increases the hiding power relative to the pure pigment, but impairs the flop/Alman Index).

It is also possible to mix a silver dollar pigment available commercially from Schlenk Metallic Pigments GmbH under the designation “Alustar 10200”, with 4% pigmentation, with 4.5% (initial masses of the yellow pigment, unless otherwise characterized, based on the dry mass of the pigment used) of a yellow pigment (Helio Beit UN120), available commercially from Helio Beit Pigmentpasten GmbH, in order to obtain a color tone in the region of the pigments of the invention. For both, in fact, the ΔE45° hiding power of a mixture obtained in this way is within the range achieved by the metallic effect pigments of the invention. Flop and Gdiff of the mixture are likewise in the range of the metallic effect pigments of the invention. However, with a value for the saturation (Sᵤᵥ) of around 0.5 and with a value of around 70 for the hue angle (hᵤᵥ), it is not possible to attain the ranges of the metallic effect pigments of the invention.

If the proportion of the yellow pigment in the mixture above is increased from 4.5% to 10%, it is indeed possible to meet the range for the hue angle (hᵤᵥ) of the metallic effect pigments of the invention, but this leads to a higher saturation, which is significantly outside the range of the metallic effect pigments of the invention.

A yellowish-beige champagne tone with a hue angle (hᵤᵥ) in the range of 50-65, with very high hiding power and a saturation (Sᵤᵥ) in the range of 0.2-0.3, cannot be generated in this combination by way of mixtures of pearlescent pigments and/or metallic effect pigments with one another or through addition of carbon black or dyes. With an approximately equal color tone, there are also differences in the flop. It is often necessary to add carbon black for improving the hiding power.

Further disadvantages of mixtures of silver-color pigments based, for example, on silver dollars with organic dyes, e.g., with an organic yellow pigment, are as follows: UV stabilities and temperature stabilities are lower, an additional mixing procedure is necessary, and in some instances it is necessary to add stabilizers or emulsifiers.

Colorations of Al pigments with the more UV-stable inorganic (yellow) pigments do not achieve the corresponding brightness of an Al pigment with a corresponding iron oxide layer (literature regarding the photochemical decomposition of organic pigments: Lisa Ghelardi, Ilaria Degano, Maria Perla Colombini, Joy Mazurek, Michael Schilling, Herant Khanjian, Tom Learner, A multi-analytical study on the photochemical degradation of synthetic organic pigments, Dyes and Pigments, Volume 123, 2015, pages 396-403).

Nor can the desired color tone with a saturation (Sᵤᵥ) in the range of 0.2-0.3 be achieved by mixing a gold-color aluminum pigment (selected illustratively, the pigment 21YY (Zenexo Goldenshine 21 YY from Schlenk Metallic Pigments GmbH) having the following pigment construction: substrate=Decomet (VMP aluminum platelet) with layer thickness ˜25 nm, thereon an SiO₂ layer with ˜60 nm, and lastly an Fe₂O₃ layer with ˜100 nm, with further SiO₂ coating of ˜10 nm) with a carbon black, TiO₂ or a pearlescent pigment (here, in the example, Iriodin 119 Polar White from Merck). These formulations do have good hiding power and a good flop, but the above value for the saturation (Sᵤᵥ) cannot be achieved by way of the corresponding blends.

The hiding power is determined by varying the pigmentation of the paint mixtures and ascertaining the pigmentation that is needed for a knife-applied hiding coating at a predetermined wet film thickness. The pigmentation (in %) here refers to the mass of the pure pigment as a proportion of the total mass of the paint mixture in a solvent-based nitrocellulose/polycyclohexanone/polyacrylic varnish (SC-10%). For this determination, at least four different pigmentations are applied with a wet film thickness of 38 μm to a black/white card from TQC, using a film-thawing apparatus from Zehnter, and, after drying, the ΔE45° is recorded using a Byk-mac I instrument from Byk. From the ΔE45° values obtained, a curve is fitted and the value for the pigmentation for a ΔE45°=1 is ascertained. A layer is defined as a hiding layer if it has a ΔE45° of ≤1 at a predetermined wet film thickness of 38 μm. The lower the pigmentation needed to achieve this value, the higher the hiding power.

Generally speaking, the platelets have mean largest diameters of about 2 to 200 μm, more particularly about 5 to 100 μm. The d₅₀ of the pigments of the invention (Al substrate+SiO₂+Fe₂O₃ layer) is in general 15-30 μm. The d₅₀ value herein, unless otherwise indicated, is determined using a Sympatec Helos instrument with Quixel wet dispersing.

In general the coating of part of the surface of the coated substrate platelets is sufficient to obtain a luster pigment. For example, only the upper side and/or lower side of the platelets can be coated, with the side face(s) being left out. In accordance with the invention, however, the entire surface of the optionally passivated substrate platelets, including the side faces, is covered by coatings A and B. The substrate platelets are therefore completely enveloped by the coatings A and B. This improves the optical properties of the pigment of the invention and increases the mechanical and chemical robustness of the coated substrate platelets.

The method of the invention for producing the metallic luster pigment encompasses the steps of providing aluminum substrate platelets, generally passivated aluminum substrate platelets, applying the SiO₂ layer A wet-chemically via a sol-gel process to one such aluminum substrate platelet provided beforehand, and subsequently applying the Fe₂O₃ layer B by means of wet-chemical precipitation of one or more iron(III) salts and subsequent calcining.

In order to generate coating A, organosilicon compounds in which the organic radicals are bonded to the metals via oxygen atoms are usefully hydrolyzed in the presence of the substrate platelets. A preferred example of the organosilicon compounds is tetraethoxysilane (tetraethyl orthosilicate, TEOS). The hydrolysis is carried out preferably in the presence of a base, e.g., KOH, NaOH, Ca(OH)₂ or NH₃, or an acid, for example, phosphoric acid and organic acids such as acetic acid, as catalyst. Water must be present at least in the amount needed stoichiometrically for the hydrolysis, although preference is given to 2 to 100 times, more particularly 5 to 20 times, the amount. Based on the amount of water used, it is general practice to add 1 to 40 vol %, preferably 1 to 10 vol %, of a 25 wt % strength aqueous base, e.g., KOH, NaOH, Ca(OH)₂ or NH₃. For the temperature regime it has proved advantageous to heat the reaction mixture for 1-12 h, preferably for 2-8 h, at 40-80° C., preferably at 50-70° C.

In terms of process, the coating of substrate platelets with a coating A is accomplished usefully as follows:

Aluminum substrate platelets, organic solvent, water and catalyst are introduced, after which the silicon compound for hydrolysis, as the pure substance or in solution, for example, as a 30 to 70, preferably 40 to 60 vol % strength solution in the organic solvent, is added to the preheated reaction mixture. The silicon compound can alternatively be metered in continuously at elevated temperature, in which case water and aqueous base may be introduced initially or likewise metered in continuously. After coating is at an end, the reaction mixture is cooled back down to room temperature.

To prevent agglomeration during the coating procedure, the suspension may be subjected to a strong mechanical stress such as pumping, vigorous stirring or the action of ultrasound.

The coating step may optionally be repeated one or more times. If the mother liquor has a milky cloudy appearance, it is advisable to replace it prior to further coating.

The aluminum substrate platelets clad with coating A may be isolated in a simple way by filtration, washing with organic solvent, preferably the alcohols also commonly used as solvents, and subsequent drying (typically 2 to 24 h at 20 to 200° C.).

The layer B may be applied by hydrolytic decomposition of iron(III) salts such as iron(III) chloride and sulfate, with subsequent conversion of the resulting hydroxide-containing layer into the oxide layer by calcining. Heating takes place preferably at a temperature of 250 to 550° C. for a duration of 5 to 360 minutes, preferably 300 to 450° C. for a duration of 30 of 120 minutes.

If the iron oxide layer is intended as a mixed layer, which comprises one or both of the metal oxides, selected from Al₂O₃ or TiO₂, in a (total) fraction of 0 to 20 wt %, based on the layer B, then, in addition to the iron(III) salt, for example, AlCl₃ and/or TiOCl₂ may be used as precursor compounds for Al₂O₃ and TiO₂, respectively.

With the aid of the production method of the invention, the coated substrate platelets can be reproducibly produced in a simple way in large quantities. Fully enveloped pigment particles are obtained, with a high quality of the individual coatings (homogeneous, filmlike).

The metallic effect pigments of the invention can be used advantageously in cosmetics, provided they are toxicologically/microbiologically unobjectionable. Broad-spectrum usage is possible here: primarily nail varnishes, lip gloss, eyeshadow, lipsticks. Further possible uses include, for example, shampoos and powders.

To boost the effect, the pigment of the invention may be mixed with organic or inorganic dyes.

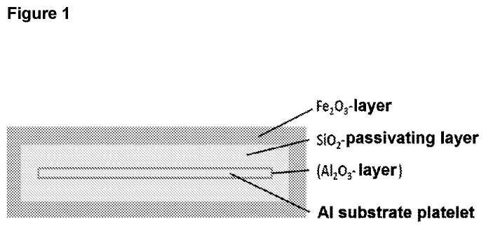

FIG. 1 shows the general layer construction of the metallic effect pigments of the invention (not to scale).

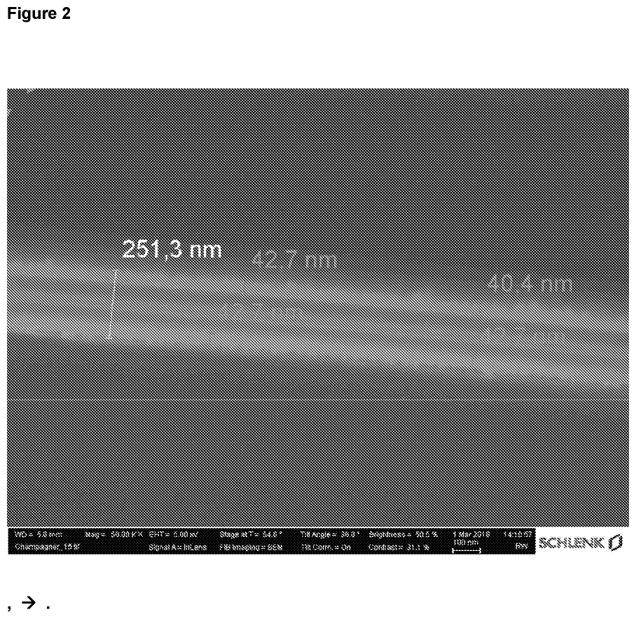

FIG. 2 shows the SEM section of a metallic effect pigment of the invention, namely the inventive pigment (construction of this pigment: substrate=Decomet (VMP aluminum platelet) with layer thickness ˜25 nm, thereon an SiO₂ layer with ˜60 nm, and lastly an Fe₂O₃ layer with ˜45 nm, without a further coating, such as a further SiO₂ coating, for example, and without surface modification).

The examples which follow serve as a further illustration of the present invention, without being confined thereto.

### EXAMPLES

Inventive pigment=Decomet (VMP aluminum) as substrate, layer A=SiO₂ with layer thickness in the inventive range, layer B=Fe₂O₃ with layer thickness in the inventive range.

### Inventive Examples

(1) Inventive pigment+around 1.6% Al₂O₃
(2) Inventive pigment
(3) Inventive pigment with around 6% TiO₂
(4) Inventive pigment with around 1.6% Al₂O₃+SiO₂ (layer C, arithmetic thickness 16.5 nm)
(5) Inventive pigment with around 1.6% Al₂O₃+SiO₂ (layer C, arithmetic thickness 26.5 nm)

### Noninventive Examples

(6) Merck Iriodin 9602 with 4.5% yellow pigment Helio Beit UN 210
(7) Merck Iriodin 9602
(8) Alustar 10200 with 4% yellow pigment Helio Beit UN 210
(9) Alustar 10200 with 10% yellow pigment Helio Beit UN 210
(10) Pigment 21 YY (Zenexo GoldenShine, Schlenk Metallic Pigments GmbH)
(11) Pigment 21YY with 1% Helio Beit Carbon Black (carbon black dispersion)
(12) Pigment 21YY with 10% Iriodin 119
(13) Pigment 21YY with 20% Iriodin 119
(14) Pigment 21YY with 50% Iriodin 119
(15) Pigment 21YY with 1.5% TiO₂

### Production of Inventive Examples

First of all, Al platelets were coated with SiO₂ by a sol-gel method using tetraethyl orthosilicate (TEOS) (cf., for example, WO 2015/014484 A1).

Subsequently a 2 L beaker is charged with the amount of suspension corresponding to an aluminum content of 15 g. The suspension is made up with mains water to 900 g.

At the start of the synthesis, slight acidification is carried out. This is followed by commencement of addition of the FeCl₃ solution; the pH is kept constant by the addition of an alkaline solution, e.g., KOH, NaOH, Ca(OH)₂ or NH₃. At the end of the synthesis the pH is raised with stirring. Subsequently, after sedimentation, the resulting pigment is separated off by filtration, dried, and subsequently calcined.

The table below reports the layer thicknesses and coloristic data for above inventive and noninventive examples:

| | | | | Hiding power | | | | | |
|---|---|---|---|---|---|---|---|---|---|
| | | Hue angle | Saturation | ΔE45° | | % pigmentation | Alman | | |
| Designation of the examples | No. | (hᵤᵥ) | (Sᵤᵥ) | (% pigmentation) | | for ΔE45° = 1 | Index | Gdiff | Inventive |
| Inventive range | | 50-65 | 0.2-0.3 | ≤3 | (7%) | <7% | >26 | <6 | |
| Inventive pigment | 1 | 55-57 | 0.23-0.24 | 3.4 (4.2%), | | 5.1 | 33-34 | 4.0-5.0 | Yes |
| (with around 1.6% Al₂O₃) | | | | 0.06 (7.2%) | | | | | |
| Inventive pigment | 2 | 54 | 0.26 | 0.2 | (7%) | 5.5 | 34 | 5.0 | Yes |
| Inventive pigment | 3 | 61 | 0.20 | 2.4 (5%), | | 5.7 | 34 | 4.7-5.3 | Yes |
| (with around 6.0% TiO₂) | | | | 0.2 (7%) | | | | | |
| Inventive pigment | 4 | 56 | 0.26 | 2.4 | (5%) | 6.2 | 33.6 | 5.2 | Yes |
| (with around 1.6% Al₂O₃) + 16.5 nm | | | | | | | | | |
| (calculated) SiO₂ (layer C) | | | | | | | | | |
| Inventive pigment | 5 | 56 | 0.26 | 2.8 | (5%) | 6.7 | 33.2 | 5.3 | Yes |
| (with around 1.6% Al₂O₃) + 26.5 nm | | | | | | | | | |
| (calculated) SiO₂ (layer C) | | | | | | | | | |
| Merck Iriodin 9602 + | 6 | 71 | 0.26 | 8.4 | (20%) | — | 22 | 3.8 | No |
| 4.5% yellow pigment Helio Beit UN 210 | | | | | | | | | |
| Merck Iriodin 9602 | 7 | 238-239 | 0.10-0.13 | 54 (4.2%), | | 31.8 | 27-31 | 5.0-6.5 | No |
| | | | | 35 (7.2%) | | | | | |
| Alustar 10200 + | 8 | 71 | 0.48 | 1.6 | (4%) | — | 28 | 5.4 | No |
| 4% yellow pigment Helio Beit UN 210 | | | | | | | | | |
| Alustar 10200 + | 9 | 65 | 0.70 | 1.2 | (4%) | — | 26 | 5.5 | No |
| 10% yellow pigment Helio Beit UN 210 | | | | | | | | | |
| 21 YY (Zenexo GoldenShine) | 10 | 62 | 0.92 | 1.8 | (7%) | 7.8 | 31 | 5.0 | No |
| 21 YY (Zenexo GoldenShine) + | 11 | 63 | 0.89 | 2.2 | (7%) | — | 31 | 5.2 | No |
| 1% Helio Beit Carbon Black | | | | | | | | | |
| 21 YY (Zenexo GoldenShine) + | 12 | 63 | 0.83 | 3.1 | (7%) | 9.1 | 29 | 5.1 | No |
| Merck Iriodin 119 10% | | | | | | | | | |
| 21 YY (Zenexo GoldenShine) + | 13 | 63 | 0.75 | 4.4 | (7%) | 9.6 | 27 | 5.0 | No |
| Merck Iriodin 119 20% | | | | | | | | | |
| 21 YY (Zenexo GoldenShine) + | 14 | 62 | 0.50 | 10.2 | (7%) | 13.2 | 22 | 4.7 | No |
| Merck Iriodin 119 50% | | | | | | | | | |
| 21 YY (Zenexo GoldenShine) + | 15 | 63 | 0.89 | 2.4 | (7%) | — | 28 | 5.1 | No |
| TiO₂ 1.5% | | | | | | | | | |

The SEM micrographs were each recorded with a Zeiss Auriga 40 scanning electron microscope.
The coloristic data were produced by measuring knife-applied coatings of the pigments (unless otherwise remarked, with a pigmentation of 7% in a solvent-based nitrocellulose/polycyclohexanone/polyacrylic varnish, SC ~10%) on a DIN A5 black/white card from TQC using a 38 μm wire-wound doctor knife on an automatic film-drawing apparatus from Zehntner.
The coloristic data were measured using the Byk-mac I from Byk.

The listing of claims replaces all previous versions of the claims:

1. A metallic effect pigment based on coated aluminum substrate platelets, where the aluminum substrate platelets have a thickness of 5 to 35 nm, are of monolithic construction, and are surrounded by a native passivation layer of Al₂O₃ of less than 10 nm, where these aluminum substrate platelets are enveloped by at least one coating A of SiO₂ having a layer thickness of 10 to 100 nm, bearing a further, applied coating B of Fe₂O₃ which envelops the substrate platelets having the SiO₂ coating A and which has a layer thickness of 5 to 45 nm, and characterized, for a ΔE45 of ≤3 at not more than 7% pigmentation, more particularly ΔE45° of ≤1 at <7% pigmentation, by a saturation (Sᵤᵥ) in the range of 0.2-0.3, a value for the hue angle (hᵤᵥ) in the range of 50-65, a value for the flop/Alman Index above 26, and a value for the Gdiff (graininess) at not more than 6.

2. The metallic effect pigment as claimed in claim 1, wherein the aluminum substrate platelets have a thickness of 15 to 25 nm.

3. The metallic effect pigment as claimed in claim 1, wherein the SiO₂ coating A has a layer thickness of 20 to 80 nm.

4. The metallic effect pigment as claimed in claim 1, wherein the Fe₂O₃ coating B has a layer thickness of 10 to 45 nm.

5. The metallic effect pigment as claimed in claim 1, wherein the iron oxide layer is a mixed layer which comprises one or both of the metal oxides, selected from Al₂O₃ or TiO₂, in a fraction of more than 0 to 20 wt %, based on the layer B.

6. The metallic effect pigment as claimed in claim 1, further comprising an outer layer C, which in turn envelops the coating B, more particularly an SiO₂ layer 1 to 30 nm thick, which optionally may also penetrate the lower Fe₂O₃.

7. The metallic effect pigment as claimed in claim 1, wherein an organic or organic-inorganic surface modification has been applied to the layer B or to the layer C, if present.

8. A method for producing a metallic effect pigment as claimed in claim 1, comprising applying the SiO₂ layer A wet-chemically via a sol-gel process to a pre-provided aluminum substrate platelet and subsequently applying the Fe₂O₃ layer B by means of wet-chemical precipitation of one or more iron(III) salts and subsequent calcining.

9. The method as claimed in claim 8, further comprising applying a further, outer layer C, which in turn envelops the coating B, more particularly an SiO₂ layer 1 to 30 nm thick, which optionally may also penetrate the lower Fe₂O₃.

10. The method as claimed in claim 8, further comprising applying an organic or organic-inorganic surface modification to the layer B or to the layer C, if present.

11. A coloring paint, printing ink, liquid ink, plastic, glass, ceramic product, or preparation of decorative cosmetic, especially in nail varnishes, lip gloss, eyeshadow, lipsticks, shampoos and/or powders, comprising the metallic effect pigments of claim 1, where the metallic effect pigments is used alone or in a mixture with an organic and/or inorganic pigment.
